(19) United States
(12) Patent Application Publication
(10) Pub. No.: US 2018/0049632 A1
(43) Pub. Date: Feb. 22, 2018

(54) ENDOSCOPE APPARATUS

(71) Applicant: OLYMPUS CORPORATION, Tokyo (JP)

(72) Inventor: Hiromi SHIDA, Tokyo (JP)

(73) Assignee: OLYMPUS CORPORATION, Tokyo (JP)

(21) Appl. No.: 15/786,675

(22) Filed: Oct. 18, 2017

Related U.S. Application Data
(63) Continuation of application No. PCT/JP2015/062149, filed on Apr. 21, 2015.

(51) Int. Cl. A61B 1/06; A61B 1/045; A61B 1/00

(52) U.S. Cl. CPC A61B 1/0638; A61B 1/045; A61B 1/00045; A61B 1/00039; A61B 1/00009

(57) ABSTRACT

An endoscope apparatus includes: an illumination unit that sequentially radiates illumination light of three colors of RGB onto a subject; an image acquisition unit that captures the illumination light reflected at the subject; a control unit that controls the image acquisition unit so as to sequentially capture the illumination light of the three colors and so as to capture the illumination light of at least one color other than G, a plurality of times for different exposure times, thereby causing the image acquisition unit to acquire component images of the three colors; a dynamic-range expanding unit that generates an expanded component image by compositing the plurality of component images of the at least one color; and an image generating unit that generates a colored endoscope image by compositing the expanded component image of the at least one color and the component images of the other colors.

### CROSS-REFERENCE TO RELATED APPLICATIONS

This is a continuation of International Application PCT/JP2015/062149, with an international filing date of Apr. 21, 2015, which is hereby incorporated by reference herein in its entirety. This application claims the benefit of International Application PCT/JP2015/062149, the content of which is incorporated herein by reference.

### TECHNICAL FIELD

The present invention relates to an endoscope apparatus.

### BACKGROUND ART

There are conventionally known endoscope apparatuses in which a subject is captured a plurality of times for different exposure times, and a plurality of acquired images are composited, thereby acquiring an endoscope image in which the dynamic range has been expanded (for example, see PTL 1).

In an endoscope apparatus of PTL 1, a color CCD is used to capture a subject two times, namely, for a long exposure time ( 1/60 seconds) and for a short exposure time ( 1/240 seconds), and two acquired digital signals are divided into signals of three colors: R, G, and B. Next, an R signal during the short exposure time and the R signal during the long exposure time are composited, thereby generating an R signal in which the dynamic range has been expanded. The dynamic ranges of the G signal and the B signal are expanded in the same way as the R signal. Next, the R signal, the G signal, and the B signal, in which the dynamic ranges have been expanded, are used to generate a color endoscope image having a dynamic range wider than the dynamic range of the CCD.

In capturing performed for a short exposure time, a bright area, such as a near-point area, onto which strong illumination light is radiated is clearly captured without causing halation. In capturing performed for a long exposure time, a dark area, such as a far-point area, that illumination light is unlikely to reach is clearly captured without causing underexposed shadows. Therefore, the endoscope apparatus of PTL 1 is suitable for capturing a subject in which the difference in brightness is large, such as a tubular digestive tract.

### CITATION LIST

### Patent Literature

{PTL 1} Japanese Unexamined Patent Application, Publication No. Hei 11-234662

### SUMMARY OF INVENTION

### Technical Problem

In endoscope-image diagnosis, an observer pays attention to an inflamed portion in red, such as redness, and veins in blue. Furthermore, a method in which a blue dye, which has high contrast with respect to the color of reddish living tissue, is sprayed on an area to be diagnosed to emphasize the structure of the area to be diagnosed in the living tissue is used in some cases. In this way, endoscope images tend to have red or blue color in many cases, and information concerning red and blue is particularly important in endoscope-image diagnosis.

An object of the present invention is to provide an endoscope apparatus capable of acquiring an endoscope image in which the difference in color of living tissue is correctly reproduced.

### Solution to Problem

An aspect of the present invention provides an endoscope apparatus including: an illumination unit that sequentially radiates illumination light of three colors of red, green, and blue onto a subject; an image acquisition unit that acquires an image by capturing the illumination light reflected at the subject; a control unit that controls the image acquisition unit so as to perform capturing in synchronization with radiation of the illumination light of the three colors from the illumination unit, thereby causing the image acquisition unit to sequentially acquire component images of three colors of red, green, and blue; a dynamic-range expanding unit that generates an expanded component image in which the dynamic range is expanded, from the component image of at least one color other than green, among the component images of the three colors acquired by the image acquisition unit; and an image generating unit that generates a colored endoscope image by compositing the expanded component image of the at least one color, which is generated by the dynamic-range expanding unit, and the component images of the other colors, wherein the control unit controls the image acquisition unit so as to capture the illumination light of the at least one color a plurality of times for different exposure times, thereby causing the image acquisition unit to acquire a plurality of component images of the at least one color; and the dynamic-range expanding unit generates the expanded component image by compositing the plurality of component images of the at least one color.

In the above-described aspect, the control unit may control the image acquisition unit so as to capture each of the illumination light of red and the illumination light of blue a plurality of times for different exposure times and may control the exposure times for capturing the illumination light of red and the exposure times for capturing the illumination light of blue, independently from each other.

The above-described aspect may further include an exposure-time setting unit that sets exposure times for next capturing of the illumination light of the at least one color performed a plurality of times, on the basis of the distribution of gradation values of the plurality of component images of the at least one color.

The above-described aspect may further include a region-of-interest specifying unit that specifies a region of interest in a capture range of the component image captured by the image acquisition unit, wherein the exposure-time setting unit may set exposure times for next capturing of the illumination light of the at least one color performed a plurality of times, on the basis of the distribution of gradation values in the region of interest specified by the region-of-interest specifying unit, among the plurality of component images of the at least one color.

### BRIEF DESCRIPTION OF DRAWINGS

FIG. 1 is a view showing the overall configuration of an endoscope apparatus according to a first embodiment of the present invention.

FIG. 2 is a front view of a color filter set in an illumination unit of the endoscope apparatus shown in FIG. 1.

FIG. 3 is a timing chart showing the timings of radiation of illumination light and exposure performed in an image acquisition device.

FIG. 4 is a view for explaining processing performed by a dynamic-range expanding unit and a compression unit of the endoscope apparatus shown in FIG. 1.

FIG. 5 is a flowchart showing the operation of the endoscope apparatus shown in FIG. 1.

FIG. 6 is a view showing the configuration of an image processor of an endoscope apparatus according to a second embodiment of the present invention.

FIG. 7 shows: an example endoscope image (at an upper part); an image signal obtained during a long exposure time, along the line A-A of the endoscope image (at a middle part); and an image signal obtained during an extended long exposure time (at a lower part).

FIG. 8 shows: an example endoscope image (at an upper part); an image signal obtained during a short exposure time, along the line A-A of the endoscope image (at a middle part); and an image signal obtained during a shortened short exposure time (at a lower part).

FIG. 9 is a graph showing the relationship between the brightness of a subject and the gradation value of an image signal obtained during a long exposure time.

FIG. 10 is a graph showing the relationship between the brightness of a subject and the gradation value of an image signal obtained during a short exposure time.

FIG. 11 is a graph showing the relationship between the number of pixels that have the maximum gradation value and an extension time for the long exposure time.

FIG. 12 is a graph showing the relationship between the number of pixels that have the minimum gradation value and a reduction time for the short exposure time.

FIG. 13 is a flowchart showing the operation of the endoscope apparatus that is provided with the image processor shown in FIG. 6.

FIG. 14 is a flowchart showing an exposure-time setting routine shown in FIG. 13.

FIG. 15 is a timing chart showing the timings of radiation of illumination light and capturing performed in the image acquisition device.

FIG. 16 is a view showing the configuration of a modification of the image processor shown in FIG. 6.

FIG. 17 is a flowchart showing an exposure-time setting routine in the operation of an endoscope apparatus that is provided with the image processor shown in FIG. 16.

FIG. 18 is a view showing the configuration of an image processor in an endoscope apparatus according to a third embodiment of the present invention.

FIG. 19 shows an example endoscope image in which a region of interest is specified.

FIG. 20 is a flowchart showing an exposure-time setting routine in the operation of the endoscope apparatus that is provided with the image processor shown in FIG. 18.

### DESCRIPTION OF EMBODIMENTS

### First Embodiment

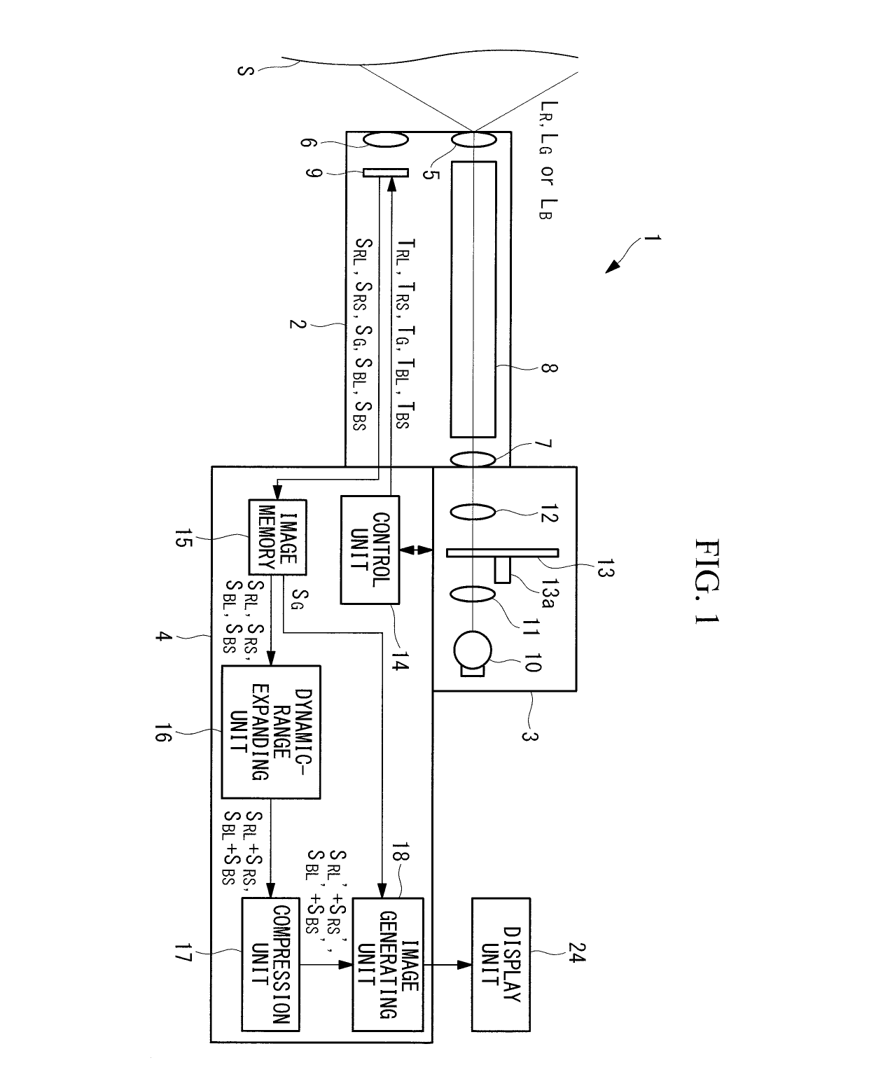

An endoscope apparatus 1 according to a first embodiment of the present invention will be described below with reference to FIGS. 1 to 5.

The endoscope apparatus 1 of this embodiment is of a frame sequential type in which illumination light of the three colors red (R), green (G), and blue (B) is sequentially radiated onto living tissue (a subject), image signals of the three colors R, G, and B are sequentially acquired, and a color endoscope image is generated from the acquired three-color image signals.

As shown in FIG. 1, the endoscope apparatus 1 is provided with: an elongated insertion portion 2 that is inserted into a living body; an illumination unit 3 that is connected to a base end of the insertion portion 2; and an image processor 4.

The insertion portion 2 is provided with: an illumination lens 5 and an objective lens 6 that are provided on a distal end surface of the insertion portion 2; a condensing lens 7 that is provided on a base end surface of the insertion portion 2; a light guide 8 that is disposed between the illumination lens 5 and the condensing lens 7 along the longitudinal direction; and an image acquisition device (image acquisition unit) 9 that is disposed at a base end side of the objective lens 6.

The condensing lens 7 focuses illumination light entering from the illumination unit 3, on a base end surface of the light guide 8.

The light guide 8 guides the illumination light incident on the base end surface thereof from the condensing lens 7 to a distal end surface thereof and emits the illumination light from the distal end surface toward the illumination lens 5.

The illumination lens 5 spreads the illumination light entering from the light guide 8 to radiate it onto living tissue S.

The objective lens 6 images, on an imaging surface of the image acquisition device 9, the illumination light reflected at the living tissue S and entering the objective lens 6.

The image acquisition device 9 is a monochrome CCD image sensor or a monochrome CMOS image sensor. As will be described later, the image acquisition device 9 is controlled by a control unit 14 so as to perform capturing in synchronization with the radiation of illumination light L_{R}, L_{G}, and L_{B} onto the living tissue S. After the end of exposure, the image acquisition device 9 generates image signals through photoelectric conversion and sends the generated image signals to an image memory 15 (to be described later) in the image processor 4.

Note that, in this embodiment, although it is assumed that the flexible insertion portion 2, in which the image acquisition device 9 is provided at a distal end portion, is used, it is also possible to use a rigid insertion portion in which a relay optical system that relays an image formed by the objective lens 6 is provided at a base end side of the objective lens 6. In the case of the rigid insertion portion, an image acquisition device is disposed at a base end side of the insertion portion.

The illumination unit 3 is provided with: a light source (for example, xenon lamp) 10 that produces white light; two condensing lenses 11 and 12 that are disposed on the output optical axis of the light source 10; and a color filter set 13 that is disposed between the two condensing lenses 11 and 12.

The condensing lens 11 focuses light produced by the light source 10 and causes the light to enter the color filter set 13. The condensing lens 12 focuses the light transmitted through the color filter set 13 and causes the light to enter the condensing lens 7 in the insertion portion 2.

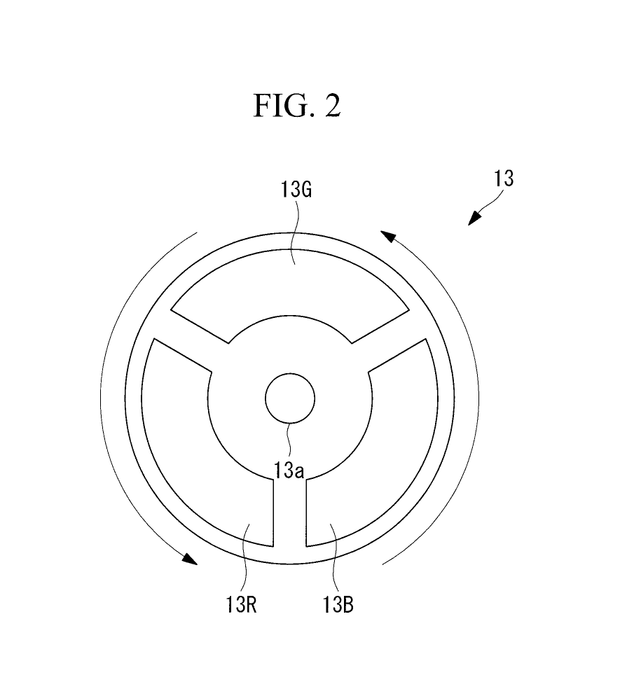

As shown in FIG. 2, the color filter set 13 has three-color filters 13R, 13G, and 13B that are evenly arranged around a rotary shaft 13a that is disposed parallel to the output optical axis of the light source 10. The R-filter 13R transmits only R-light L_{R}, the G-filter 13G transmits only G-light L_{G}, and the B-filter 13B transmits only B-light L_{B}. The color filter set 13 rotates about the rotary shaft 13a, thereby causing the filters 13R, 13G, and 13B to be sequentially disposed on the output optical axis and causing R-light L_{R}, G-light L_{G}, and B-light L_{B} to sequentially enter the condensing lens 7 from the color filter set 13.

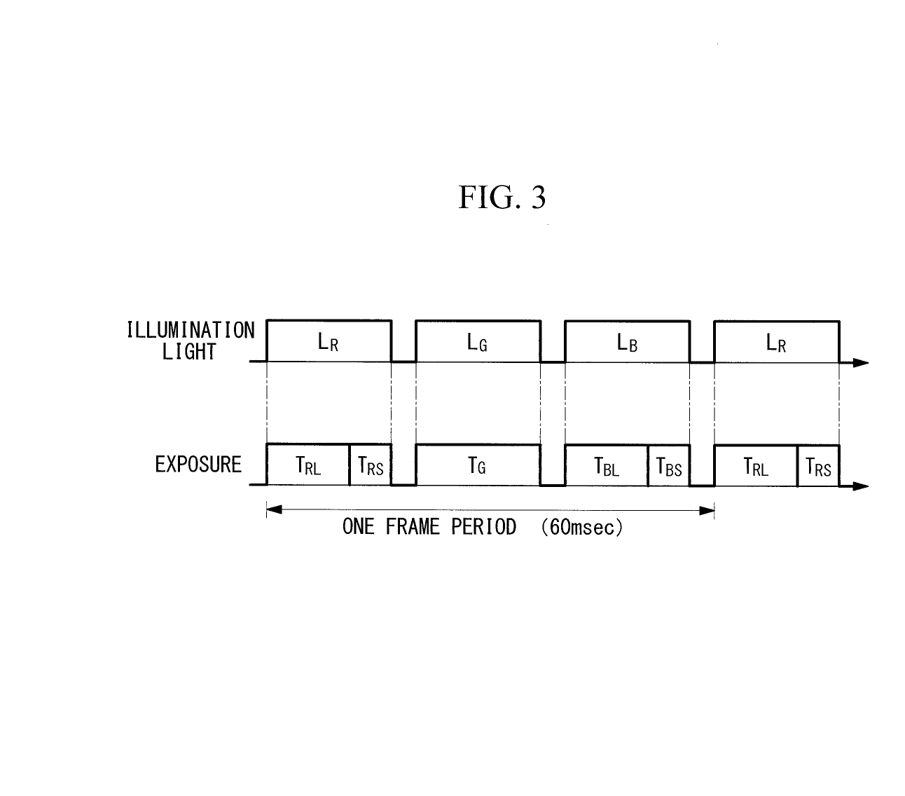

Here, the rotating speed of the color filter set 13 is fixed, and the three filters 13R, 13G, and 13B all have the same shape and dimensions. Therefore, as shown in FIG. 3, from the illumination lens 5, R-light L_{R}, G-light L_{G}, and B-light L_{B} are sequentially radiated onto the living tissue S at certain time intervals, and the irradiation times for the B-light L_{R}, the G-light L_{G}, and the B-light L_{B} per single irradiation are equal to each other. It is preferred that the rotating speed of the color filter set 13 be 30 rps or more and 60 rps or less such that the frame rate of endoscope images falls within the range from 30 fps to 60 fps, which is suitable for video.

The image processor 4 is provided with: the control unit 14, which controls the image acquisition device 9; the image memory 15, which temporarily holds image signals S_{RL}, S_{RS}, S_{G}, S_{BL}, and S_{BS} received from the image acquisition device 9; a dynamic-range expanding unit 16 that performs dynamic-range expansion processing on R-image signals S_{RL} and S_{RS} and B-image signals S_{BL} and S_{BS}; a compression unit 17 that compresses the gradation values of an R expanded image signal S_{RL}+S_{RS} and a B expanded image signal S_{BL}+S_{BS} in each of which the dynamic range has been expanded; and an image generating unit 18 that generates an endoscope image from the image signals S_{RL}′+S_{RS}′, S_{G}, and S_{BL}′+S_{BS}′.

The control unit 14 obtains, from the illumination unit 3, information of the timing of radiation of R-light L_{R}, G-light L_{G}, and B-light L_{B}. The control unit 14 causes the image acquisition device 9 to perform capturing for preset exposure times T_{RL}, T_{RS}, T_{G}, T_{BL}, and T_{BS}, on the basis of the obtained timing information, in synchronization with radiation of B-light L_{R}, G-light L_{G}, and B-light L_{B}, as shown in FIG. 3. Accordingly, the control unit 14 causes the image acquisition device 9 to perform capturing of the R-light L_{R}, the G-light L_{G}, and the B-light L_{B} in this order during one frame period.

Here, the control unit 14 causes the image acquisition device 9 to perform capturing only one time for the exposure time T_{G}, during the irradiation period for the G-light L_{G}. Accordingly, the image acquisition device 9 acquires one G-image signal S_{G} during one frame period.

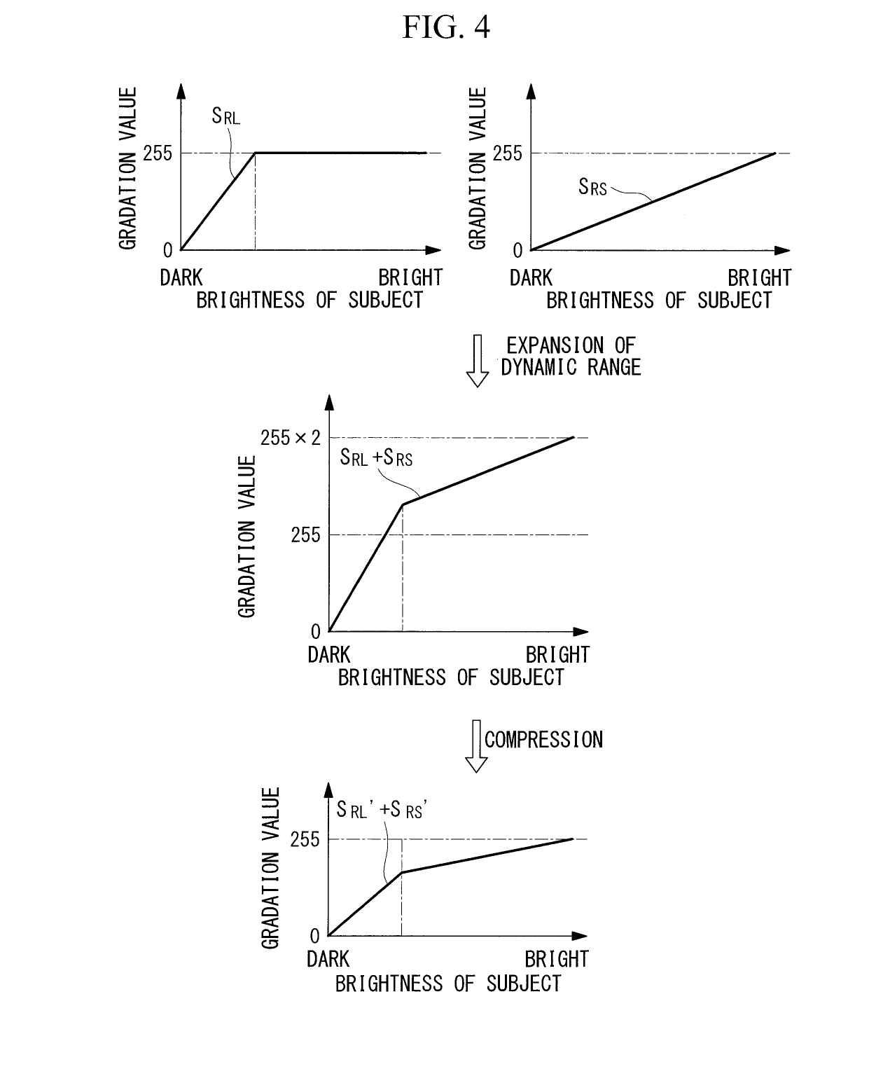

On the other hand, during the irradiation period for the R-light L_{R}, the control unit 14 causes capturing to be performed two times for the long exposure time T_{RL} and for the short exposure time T_{RS}, which is shorter than the long exposure time T_{RL}. Accordingly, the image acquisition device 9 sequentially acquires two R-image signals S_{RL} and S_{RS} having different exposure times, during one frame period. Similarly, during the irradiation period for the B-light L_{B}, the control unit 14 causes capturing to be performed two times for the long exposure time T_{BL} and for the short exposure time T_{BS}, which is shorter than the long exposure time T_{BL}. Accordingly, the image acquisition device 9 sequentially acquires two B-image signals S_{BL} and S_{BS} having different exposure times, during one frame period. As shown in FIG. 4, the image signals S_{RL} and S_{BL}, which are obtained during the long exposure times, are image signals in which dark areas of the living tissue S are clearly captured at high contrast. The image signals S_{RS} and S_{BS}, which are obtained during the short exposure times, are image signals in which bright areas of the living tissue S are clearly captured at high contrast.

The exposure times T_{RL}, T_{RS}, T_{G}, T_{BL}, and T_{BS} are set in the control unit 14 when an observer inputs desired values by using, for example, an input device (not shown) that is connected to the image processor 4. Here, the exposure times T_{RL} and T_{RS} for the R-image signals S_{RL} and S_{RS} and the exposure times T_{BL} and T_{BS} for the B-image signals S_{BL} and S_{BS} can be set independently from each other. For example, when the irradiation times for the illumination light L_{R}, L_{G}, and L_{B} per single irradiation are each 15 milliseconds, the exposure time T_{G} is set to 15 milliseconds, the long exposure times T_{RL} and T_{BL} are each set to 10 milliseconds, and the short exposure times T_{RS} and T_{BS} are each set to 5 milliseconds.

The image memory 15 sequentially receives, during one frame period, the R-image signal S_{RL}, the R-image signal S_{RS}, the G-image signal S_{G}, the B-image signal S_{BL}, and the B-image signal S_{BS}. The image memory 15 sends only the G-image signal S_{G}, which constitutes a G-component image, to the image generating unit 18 and sends the R-image signals S_{RL} and S_{RS}, which constitute an R-component image, and the B-image signals S_{BS} and S_{BL}, which constitute a B-component image, to the dynamic-range expanding unit 16.

FIG. 4 shows processing for the R-image signals S_{RL} and S_{RS} performed in the dynamic-range expanding unit 16 and the compression unit 17. Although FIG. 4 shows only the R-image signals S_{RL}, S_{RS}, S_{RL}+S_{RS}, and S_{RL}′+S_{RS}′, as example signals, the B-image signals S_{BL}, S_{BS}, S_{BL}+S_{BS}, and S_{BL}′+S_{BS}′ also have the same features.

When receiving two R-image signals S_{RL} and S_{RS} from the image memory 15, the dynamic-range expanding unit 16 adds the gradation values of respective pixels in the R-image signal S_{RL} and the gradation values of respective pixels in the R-image signal S_{RS}, thereby generating an R expanded image signal S_{RL}+S_{RS}, which constitutes an R expanded component image. Similarly, when receiving two B-image signals S_{BL} and S_{BS} from the image memory 15, the dynamic-range expanding unit 16 adds the gradation values of respective pixels in the B-image signal S_{BL} and the gradation values of respective pixels in the B-image signal S_{BS}, thereby generating a B expanded image signal S_{BL}+S_{BS}, which constitutes a B expanded component image.

The expanded image signals S_{RL}+S_{RS} and S_{BL}+S_{BS} have a dynamic range wider than the dynamic range of the image acquisition device 9 and have twice gradation scale of each of the image signals S_{RL}, S_{RS}, S_{G}, S_{BL}, and S_{BS}. The dynamic-range expanding unit 16 sends the generated R expanded image signal S_{RL}+S_{RS} and the generated B expanded image signal S_{BL}+S_{BS} to the compression unit 17.

The compression unit 17 compresses the numbers of gradations of the R expanded image signal S_{RL}+S_{RS} and the B expanded image signal S_{BL}+S_{BS} by half. Accordingly, the gradation scale of the R expanded image signal S_{RL}+S_{RS} and the B expanded image signal S_{BL}+S_{BS} become equal to the gradation scale of the G-image signal S_{G}. The compression unit 17 sends the compressed R expanded image signal S_{RL}′+S_{RS}′ and the compressed B expanded image signal S_{BL}′+S_{BS}′ to the image generating unit 18.

The image generating unit 18 performs RGB-composition on the unprocessed G-image signal S_{G}, which is received from the image memory 15, and the R expanded image signal S_{RL}′+S_{RS}′ and the B expanded image signal S_{BL}′+S_{BS}′, which are received from the compression unit 17, thereby generating a colored endoscope image. The image generating unit 18 sends the generated endoscope image to a display unit 24.

The display unit 24 sequentially displays received endoscope images.

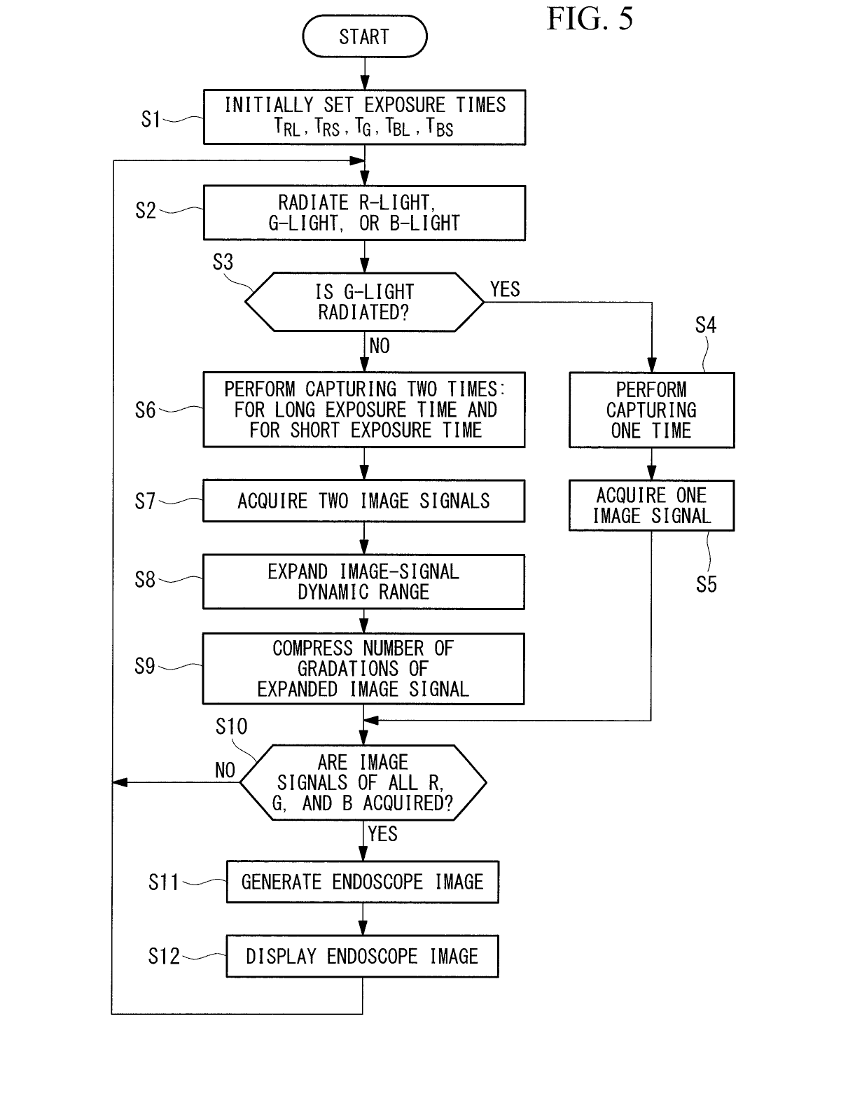

Next, the operation of the thus-configured endoscope apparatus 1 will be described with reference to FIG. 5.

First, the exposure times T_{RL}, T_{RS}, T_{G}, T_{BL}, and T_{BS} are initially set by an observer, for example (Step S1). Next, when the operation of the illumination unit 3 is started, B-light L_{R}, G-light L_{G}, and B-light L_{B} sequentially enter the light guide 8 in the insertion portion 2 via the condensing lenses 12 and 7, and the R-light L_{R}, the G-light L_{G}, and the B-light L_{B} are sequentially radiated from the distal end of the insertion portion 2 toward the living tissue S, in a repeated manner (Step S2). The R-light L_{R}, the G-light L_{G}, and the B-light L_{B} reflected at the living tissue S are collected by the objective lens 6 and are sequentially captured by the image acquisition device 9, and the image signals S_{RL}, S_{RS}, S_{GL}, S_{BL}, and S_{BS} are sequentially acquired (Steps S3 to S7).

Here, during the irradiation period for the G-light L_{G} (YES in Step S3), the control unit 14 causes the image acquisition device 9 to perform capturing only one time (Step S4), thereby acquiring one G-image signal S_{G} (Step S5).

On the other hand, during the irradiation period for the R-light L_{R} (NO in Step S3), the control unit 14 causes the image acquisition device 9 to sequentially perform capturing for the long exposure time and capturing for the short exposure time (Step S6), thereby acquiring two R-image signals S_{RL} and S_{RS} (Step S7). Similarly, during the irradiation period for the B-light L_{B} (NO in Step S3), the control unit 14 causes the image acquisition device 9 to sequentially perform capturing for the long exposure time and capturing for the short exposure time (Step S6), thereby acquiring two B-image signals S_{BL} and S_{BS} (Step S7).

The dynamic-range expanding unit 16 adds the two R-image signals S_{RL} and S_{RS} to each other, thereby generating an R expanded image signal S_{RL}+S_{RS} in which the dynamic range is expanded (Step S8). Similarly, the dynamic-range expanding unit 16 adds the two B-image signals S_{BL} and S_{BS} to each other, thereby generating a B expanded image signal S_{BL}+S_{BS} in which the dynamic range is expanded (Step S8). The gradation scale of the R expanded image signal S_{RL}+S_{RS} and that of the B expanded image signal S_{BL}+S_{BS} are compressed in the compression unit 17 (Step S9), and then, the resulting signals are sent to the image generating unit 18.

In the image generating unit 18, when the G-image signal S_{G} is input from the image acquisition device 9 via the image memory 15, and the R expanded image signal S_{RL}′+S_{RS}′ and the B expanded image signal S_{BL}′+S_{BS}′ are input from the compression unit 17 (YES in Step S10), the three-color image signals S_{G}, S_{RL}′+S_{RS}′, and S_{BL}′+S_{BS}′ are composited, thus generating a colored endoscope image (Step S11). Generated endoscope images are sequentially displayed on the display unit 24 in the form of a moving image (Step S12).

In this way, according to this embodiment, an endoscope image displayed on the display unit 24 is constituted by using the R expanded image signal S_{RL}′+S_{RS}′ and the B expanded image signal S_{BL}′+S_{BS}′, which have a wide dynamic range. Therefore, the endoscope image can correctly express deep red and deep blue without causing color saturation. Accordingly, red of an inflamed site and blue of a vein in the living tissue S, which are important in endoscope-image diagnosis, are correctly reproduced in the endoscope image, thus providing an advantage that an observer can observe, in the endoscope image, a slight change in red in the inflamed site and the detailed distribution of veins.

Furthermore, in capturing for the short exposure times T_{RS} and T_{BS}, dark areas, such as far-point areas that the illumination light L_{R}, L_{G}, and L_{B} is unlikely to reach, show underexposed shadows because the image signals S_{RS} and S_{BS} have almost no gradation values and get buried in noise; however, in capturing for the long exposure times T_{RL} and T_{BL}, the image signals S_{RL} and S_{BL} have sufficiently large gradation values in the dark areas. There is an advantage that the expanded image signals S_{RL}+S_{RS} and S_{BL}+S_{BS} are generated from these image signals S_{RL} and S_{BL}, thereby making it possible to acquire an endoscope image in which the underexposed shadows are resolved.

Furthermore, there is an advantage that the brightness of the whole endoscope image can be ensured by the G-image signal S_{G}, which is obtained during the longer exposure time T_{G} compared with the R-image signals S_{RL} and S_{RS} and the B-image signals S_{BL} and S_{BS}. Furthermore, in order to acquire an endoscope image that has high color reproducibility for red and blue, as described above, only control of the image acquisition device 9 performed by the image processor 4 and processing of the image signals S_{RL}, S_{RS}, S_{G}, S_{BL}, and S_{BS} need to be changed from those in a conventional endoscope apparatus. Therefore, there is an advantage that an endoscope image that has high color reproducibility can be acquired without complicating the configuration and while maintaining the high resolution and high frame rate of a conventional endoscope apparatus.

### Second Embodiment

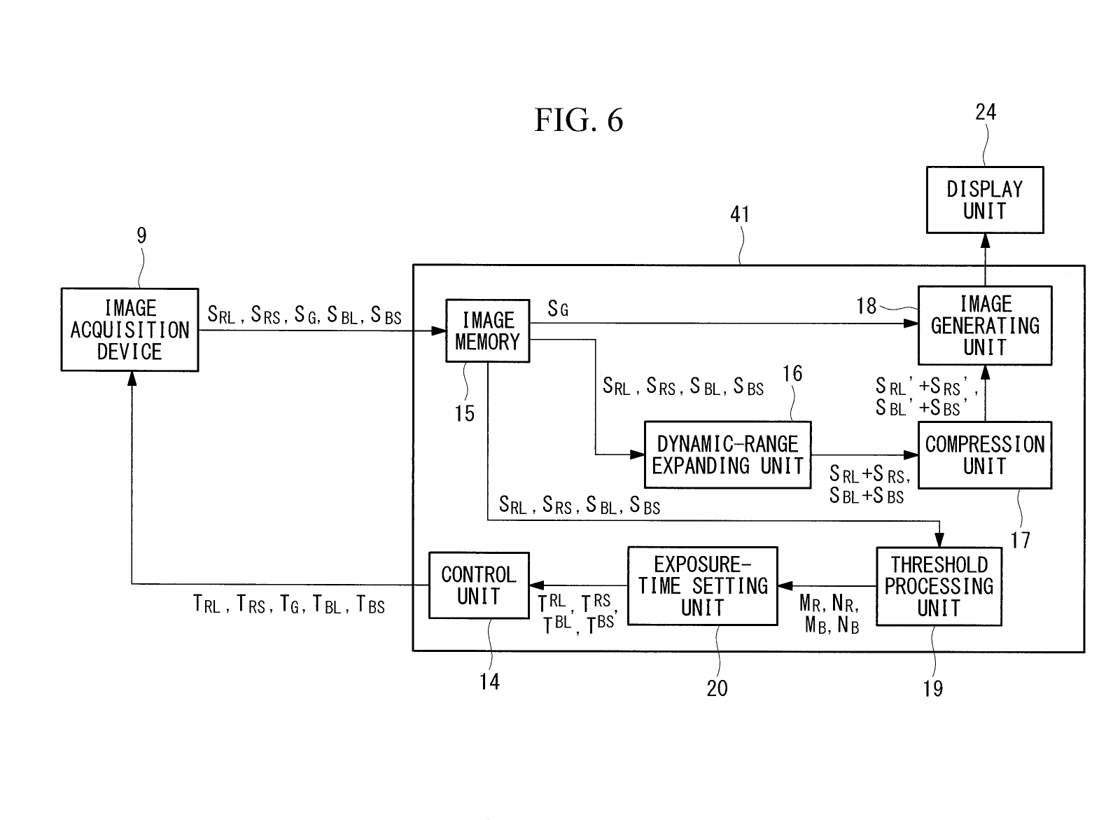

Next, an endoscope apparatus according to a second embodiment of the present invention will be described with reference to FIGS. 6 to 17.

The endoscope apparatus of this embodiment differs from the endoscope apparatus 1 of the first embodiment in that feedback control is performed on the exposure times T_{RL}, T_{RS}, T_{BL}, and T_{BS} for the next capturing of the R-light L_{R} and the B-light L_{B}, on the basis of the distributions of the gradation values of the R-image signals S_{RL} and S_{RS} and the B-image signals S_{BL} and S_{BS}.

Specifically, the endoscope apparatus of this embodiment is provided with an image processor 41 shown in FIG. 6, instead of the image processor 4. The configurations other than that of the image processor 41 are the same as those in the endoscope apparatus 1 of the first embodiment shown in FIG. 1.

As shown in FIG. 6, the image processor 41 is further provided with a threshold processing unit 19 and an exposure-time setting unit 20.

In this embodiment, the image memory 15 sends the R-image signals S_{RL} and S_{RS} and the B-image signals S_{BL} and S_{BS} to the dynamic-range expanding unit 16 and also to the threshold processing unit 19.

The threshold processing unit 19 has a threshold α_{RL} for the gradation values of the R-image signal S_{RL}, a threshold α_{RS} for the gradation values of the R-image signal S_{RS}, a threshold α_{BL} for the gradation values of the B-image signal S_{BL}, and a threshold α_{BS} for the gradation values of the B-image signal S_{BS}. The thresholds α_{RL} and α_{BL} are set to the minimum gradation value 0 of the R-image signal S_{RL} and the B-image signal S_{BL}, which are obtained during the long exposure times, or set to a value that is larger than the minimum gradation value and that is close to the minimum gradation value. The thresholds α_{BS} and α_{BS} are set to the maximum gradation value 255 of the R-image signal S_{RS} and the B-image signal S_{BS}, which are obtained during the short exposure times, or set to a value that is smaller than the maximum gradation value and that is close to the maximum gradation value.

When the R-image signal S_{RL} is received from the image memory 15, the threshold processing unit 19 measures, among all pixels of the R-image signal S_{RL}, the number of pixels M_{R} that have gradation values equal to or less than the threshold α_{RL}. Furthermore, when the R-image signal S_{RS} is received from the image memory 15, the threshold processing unit 19 measures, among all the pixels of the R-image signal S_{RS}, the number of pixels N_{R} that have gradation values equal to or greater than the threshold α_{RS}.

Similarly, when the B-image signal S_{BL} is received from the image memory 15, the threshold processing unit 19 measures, among all pixels of the B-image signal, the number of pixels M_{B} that have gradation values equal to or less than the threshold α_{BL}. Furthermore, when the B-image signal S_{BS} is received from the image memory 15, the threshold processing unit 19 measures, among all pixels of the B-image signal S_{BS}, the number of pixels N_{B} that have gradation values equal to or greater than the threshold α_{BS}.

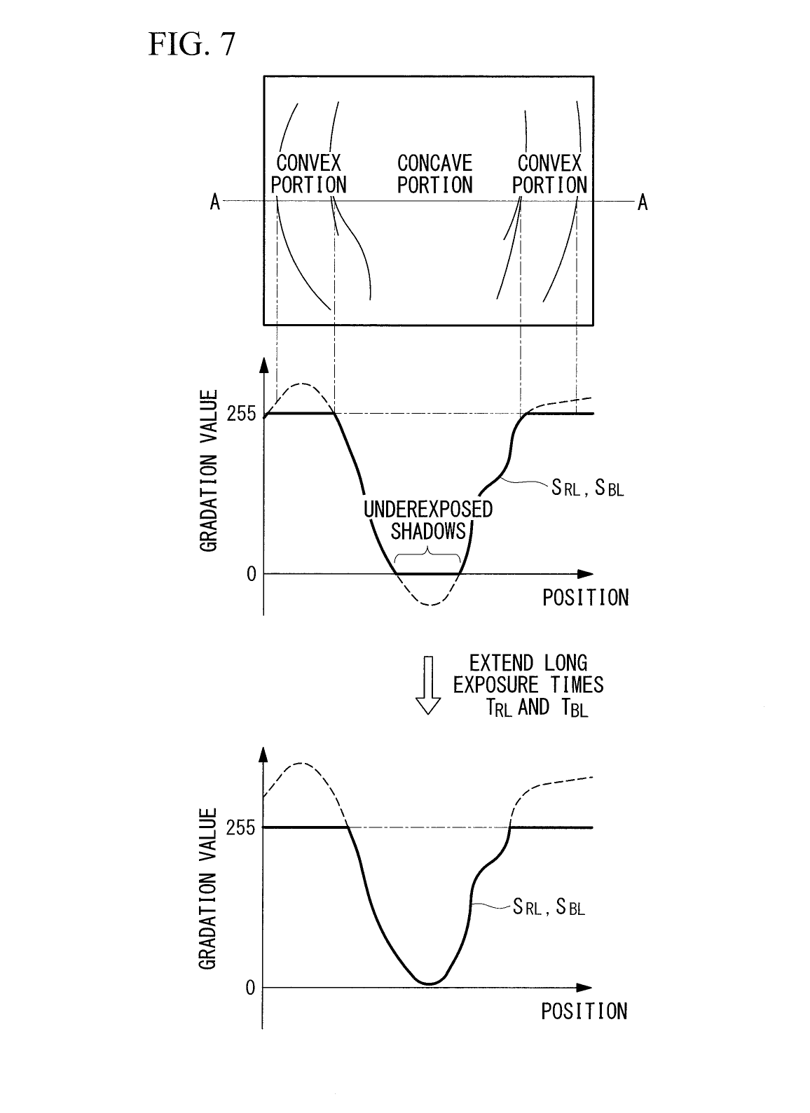

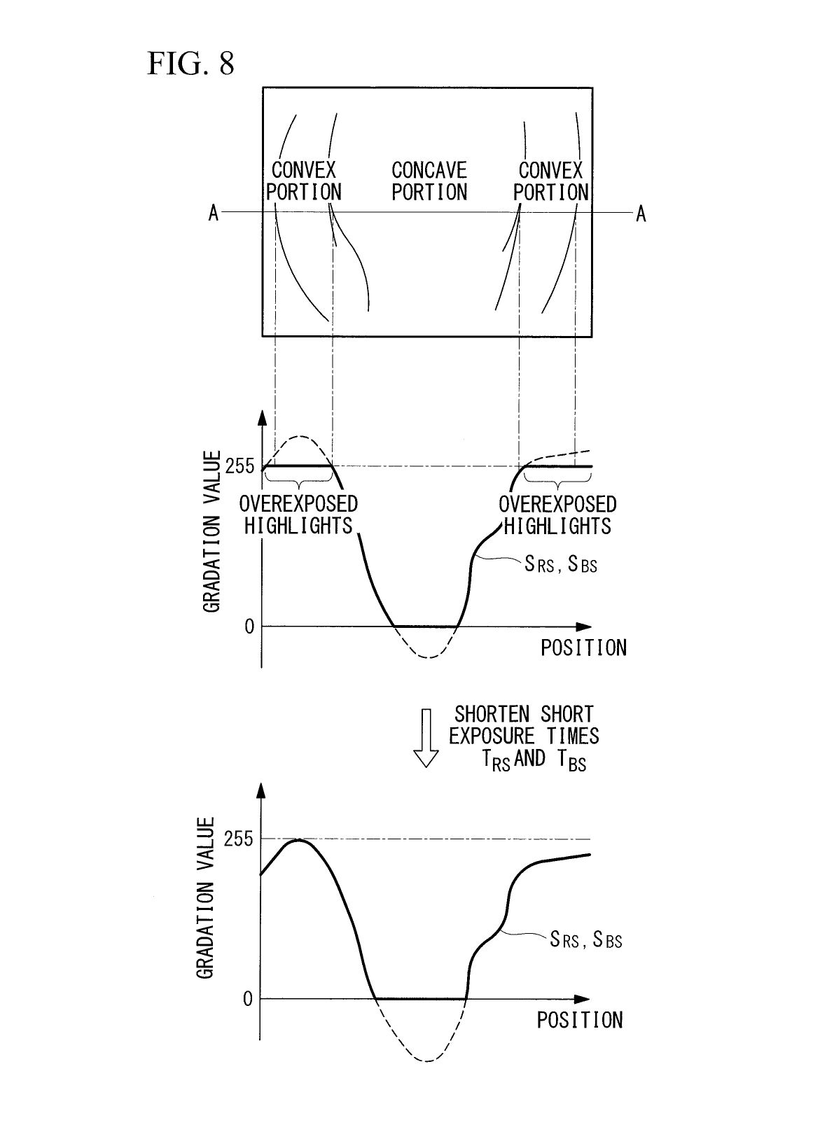

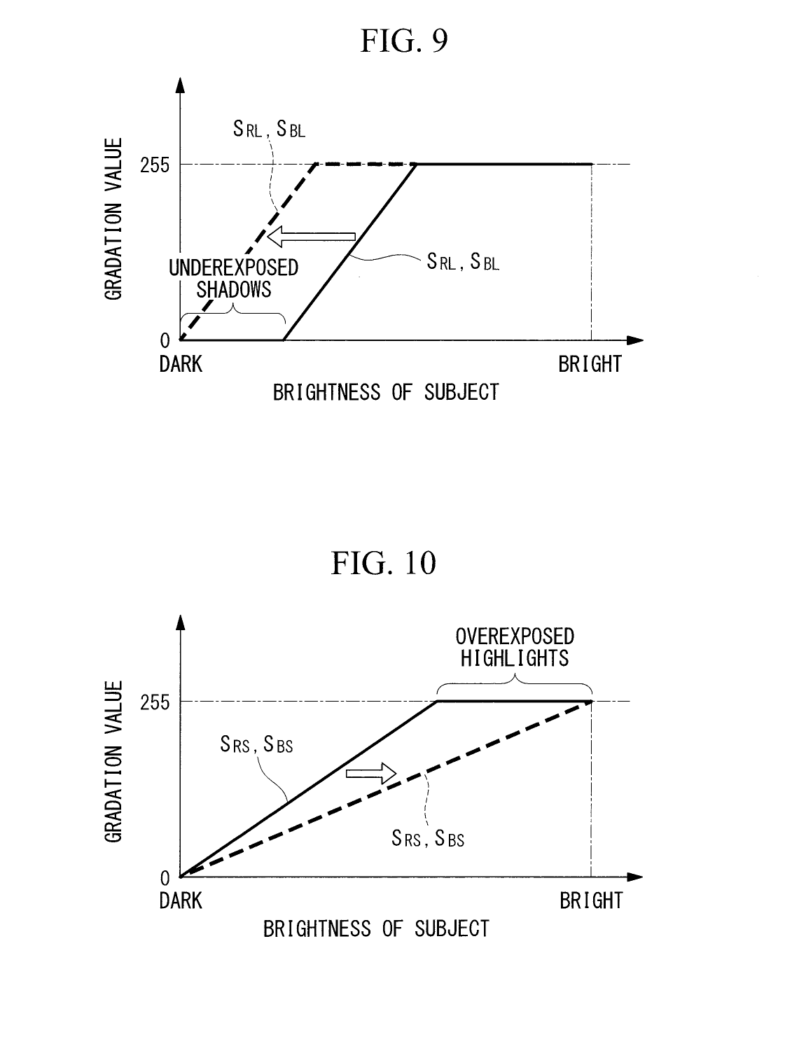

FIGS. 7 and 8 each show an example endoscope image (at an upper part) and the distributions of gradation values on the line A-A in this endoscope image (at a middle part and a lower part). FIGS. 9 and 10 show the relationships between the brightness of the living tissue S and the gradation values of the image signals S_{RL}, S_{RS}, S_{BL}, and S_{BS}.

As shown in FIGS. 7 and 8, when the living tissue S has convex portions and a concave portion, the areas of the convex portions in an endoscope image 25 become relatively bright and the area of the concave portion therein becomes relatively dark.

When the long exposure times T_{RL} and T_{BL} are too short, the exposure amounts of the illumination light L_{R} and L_{B} from the concave portion become too low, and thus, as shown in FIGS. 7 and 9, a phenomenon in which actually different darkness levels are uniformly set to the minimum gradation value 0, i.e., so-called underexposed shadows, occurs in the image signals S_{RL} and S_{BL}. In this case, the numbers of pixels M_{R} and M_{B}, which have gradation values equal to or less than the thresholds α_{RL} and α_{BL}, are increased. On the other hand, when the short exposure times T_{RS} and T_{BS} are too long, the exposure amounts in the areas of the convex portions become too high, and thus, as shown in FIGS. 8 and 10, a phenomenon in which actually different brightness levels are uniformly set to the maximum gradation value 255, i.e., so-called overexposed highlights, occurs in the image signals S_{RS} and S_{BS}. In this case, the numbers of pixels N_{R} and N_{B}, which have gradation values equal to or greater than the thresholds α_{RS} and α_{BS}, are increased.

A description will be given of an example case in which α_{RL}=α_{BL}=0, and α_{RS}=α_{BS}=255. Therefore, the threshold processing unit 19 measures the numbers of pixels M_{R} and M_{B} in an underexposed shadow area that has the minimum gradation value 0, and the numbers of pixels N_{R} and N_{B} in an overexposed highlight area that has the maximum gradation value 255.

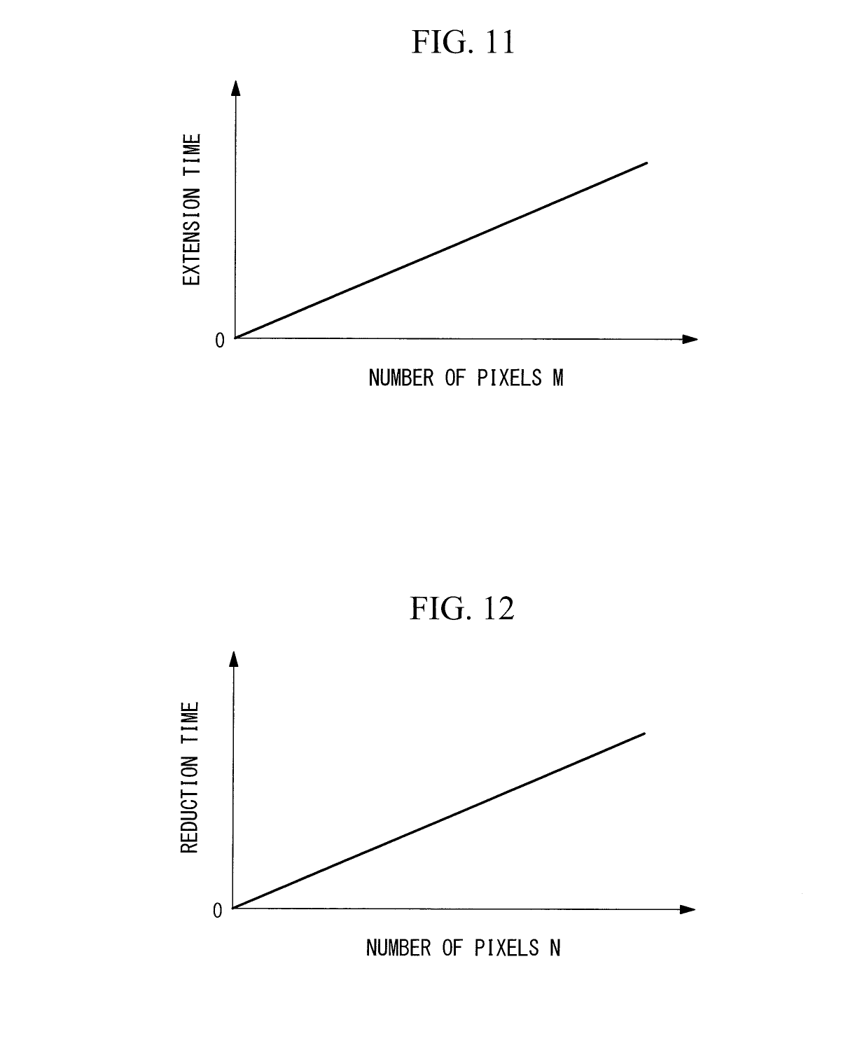

When the number of pixels M_{R} is received, the exposure-time setting unit 20 calculates an extension time for the long exposure time T_{RL} on the basis of the number of pixels M_{R} and adds the calculated extension time to the current long exposure time T_{RL}, thereby calculating a next long exposure time T_{RL}. In calculating the extension time, for example, a first look-up table (LUT) in which numbers of pixels M_{R} and extension times are associated with each other in advance is used. As shown in FIG. 11, for example, the numbers of pixels M_{R} and the extension times are associated in the first LUT such that the extension time is zero when the number of pixels M_{R} is zero, and the extension time increases in proportion to the number of pixels M_{R}.

Furthermore, when the number of pixels N_{R} is received, the exposure-time setting unit 20 calculates a reduction time for the short exposure time T_{RS} on the basis of the number of pixels N_{R} and subtracts the calculated reduction time from the current short exposure time T_{RS}, thereby calculating a next short exposure time T_{RS}. In calculating the reduction time, for example, a second LUT in which numbers of pixels N_{R} and reduction times are associated with each other in advance is used. As shown in FIG. 12, for example, the numbers of pixels N_{R} and the reduction times are associated in the second LUT such that the reduction time is zero when the number of pixels N_{R} is zero, and the reduction time increases in proportion to the number of pixels N_{R}.

The exposure-time setting unit 20 calculates, when the number of pixels M_{B} is received, a next long exposure time T_{BL} on the basis of the number of pixels M_{B}, in the same way as the long exposure time T_{RL}, and calculates, when the number of pixels N_{B} is received, a next short exposure time T_{BS} on the basis of the number of pixels N_{B}, in the same way as the short exposure time T_{RS}.

The exposure-time setting unit 20 sends the calculated next exposure times T_{RL}, T_{RS}, T_{BL}, and T_{BS} to the control unit 14.

The control unit 14 sets the exposure times T_{RL}, T_{BL}, T_{RS}, and T_{BS} received from the exposure-time setting unit 20 as exposure times for the next capturing of R-light L_{R} and B-light L_{B}.

Next, the operation of the thus-configured endoscope apparatus will be described.

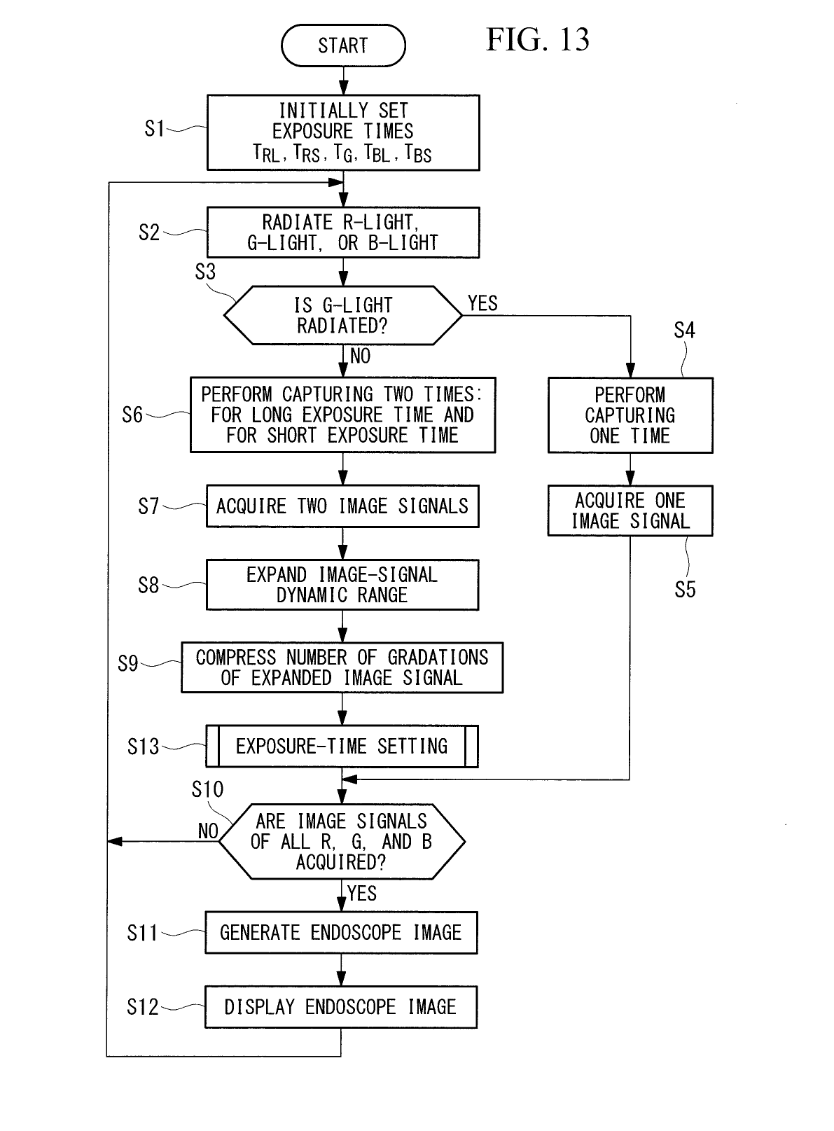

According to the endoscope apparatus of this embodiment, as shown in FIG. 13, after two R-image signals S_{RL} and S_{RS} are acquired in Step S7, the exposure times T_{RL} and T_{RS} for acquiring next R-image signals S_{RL} and S_{RS} are set on the basis of the acquired R-image signals S_{RL} and S_{RS} (Step S13).

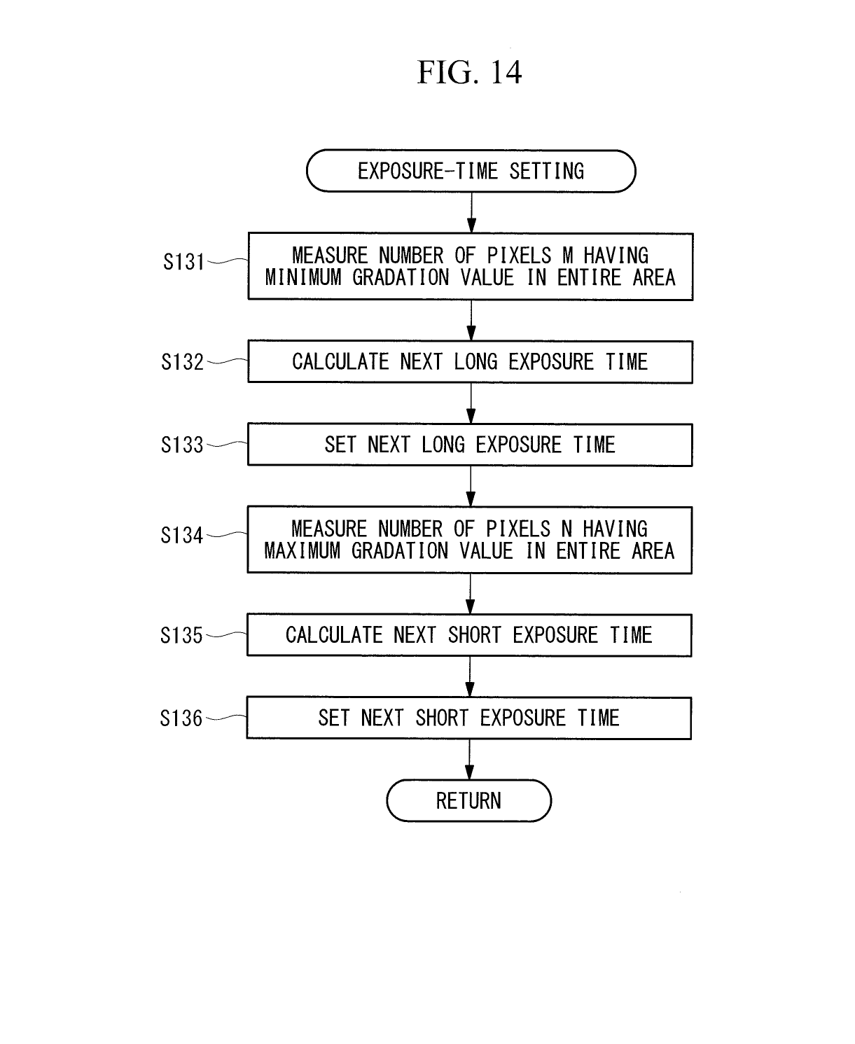

Specifically, as shown in FIG. 14, the threshold processing unit 19 measures the number of pixels M_{R} that have the minimum gradation value 0, among the all pixels of the R-image signal S_{RL}, which is obtained during the long exposure time T_{RL} (Step S131). The measured number of pixels M_{R} expresses the size of an underexposed shadow area in the R-image signal S_{RL}, and, as the underexposed shadow area becomes large, the number of pixels M_{R} is increased. The exposure-time setting unit 20 calculates a next long exposure time T_{RL} such that the next long exposure time T_{RL} becomes longer as the number of pixels M_{R} is increased (Step S132) and sets the calculated next long exposure time T_{RL} in the control unit 14 (Step S133).

Then, during the next frame period, as shown in FIG. 13, capturing of R-light L_{R} is performed for a longer long exposure time T_{RL} (Step S4). Accordingly, as shown in FIGS. 7 and 9, an image signal S_{RL} in which the underexposed shadows are resolved and that has contrast in the dark area is acquired. In a case in which no underexposed shadow area exists in the R-image signal S_{RL}, and the number of pixels M_{R} measured in Step S131 is zero, the current long exposure time T_{RL} is still calculated and set as the next long exposure time T_{RL}.

Next, the threshold processing unit 19 measures the number of pixels N_{R} that have the maximum gradation value 255, among all pixels of the R-image signal S_{RS}, which is obtained during the short exposure time T_{RS} (Step S134). The measured number of pixels N_{R} expresses the size of an overexposed highlight area in the R-image signal S_{RS}, and, as the overexposed highlight area becomes larger, the number of pixels N_{R} is increased. The exposure-time setting unit 20 calculates a next short exposure time T_{RS} such that the next short exposure time T_{RS} becomes shorter as the number of pixels N_{R} is increased (Step S135) and sets the calculated next short exposure time T_{RS} in the control unit 14 (Step S136).

Then, during the next frame period, as shown in FIG. 13, capturing of R-light L_{R} is performed for a shorter short exposure time T_{RS} (Step S4). Accordingly, as shown in FIGS. 8 and 10, an image signal S_{RS} in which the overexposed highlights are resolved and that has contrast in the bright area is acquired. In a case in which no overexposed highlight area exists in the R-image signal S_{RS}, and the number of pixels N_{R} measured in Step S134 is zero, the current short exposure time T_{RS} is still calculated and set as the next short exposure time T_{RS}.

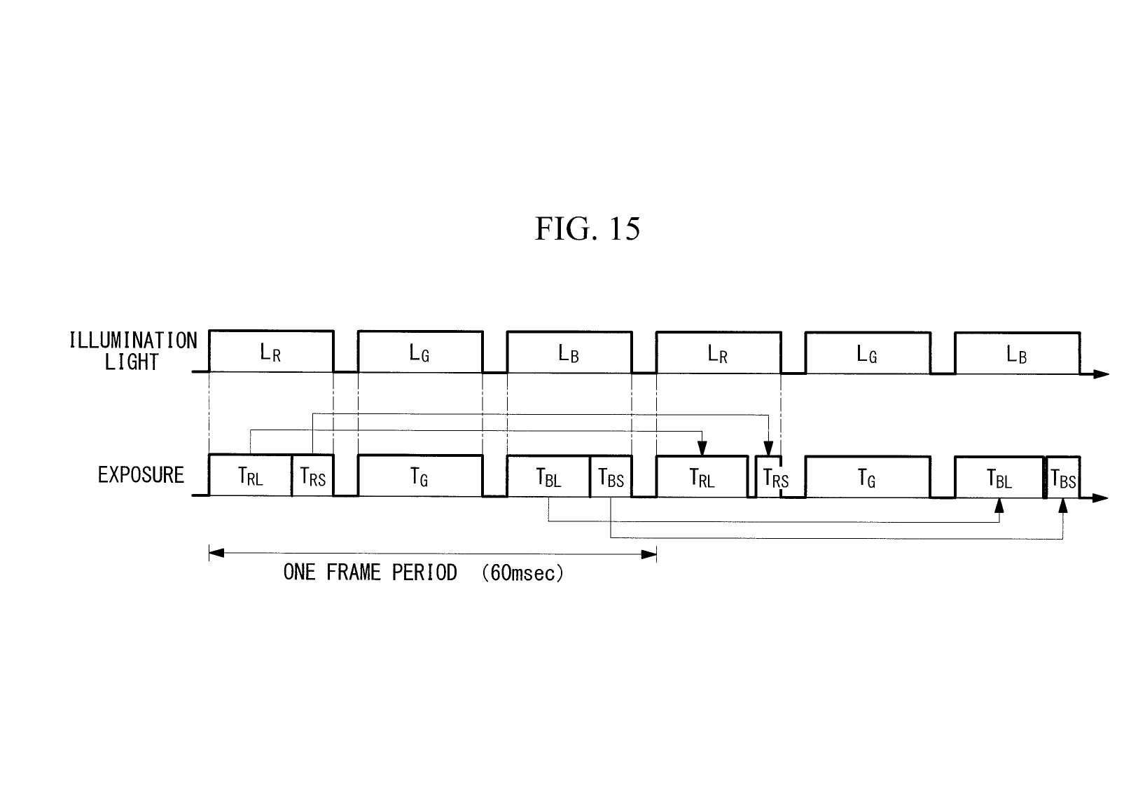

In this way, according to this embodiment, in a case in which the underexposed shadows occur because the long exposure times T_{RL} and T_{BL} are insufficient for the dark area of the living tissue S, as shown in FIG. 15, the long exposure times T_{RL} and T_{EL} for the next capturing are extended, thereby acquiring image signals S_{RL} and S_{BL} that have clear contrast in the dark area. Furthermore, in a case in which the overexposed highlights occur because the short exposure times T_{RS} and T_{BS} are excessive for the bright area of the living tissue S, as shown in FIG. 15, the short exposure times T_{RS} and T_{BS} for the next capturing are shortened, thereby acquiring image signals S_{RS} and S_{BS} that have clear contrast in the bright area.

There is an advantage in that, by using expanded image signals S_{RL}′+S_{RS}′ and S_{BL}′+S_{BS}′ that are generated from these image signals S_{RL}, S_{RS}, S_{BL}, and S_{BS}, it is possible to acquire an endoscope image in which red and blue of the living tissue S are more correctly reproduced in both the dark area and the bright area.

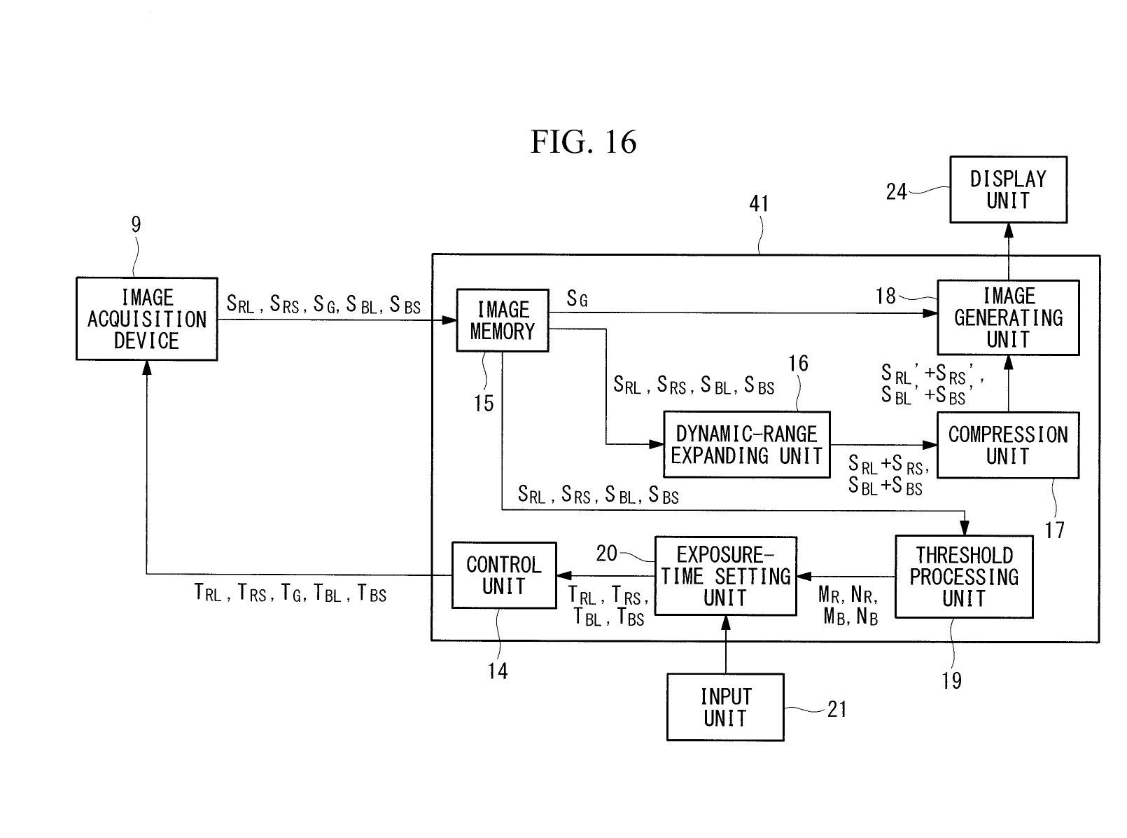

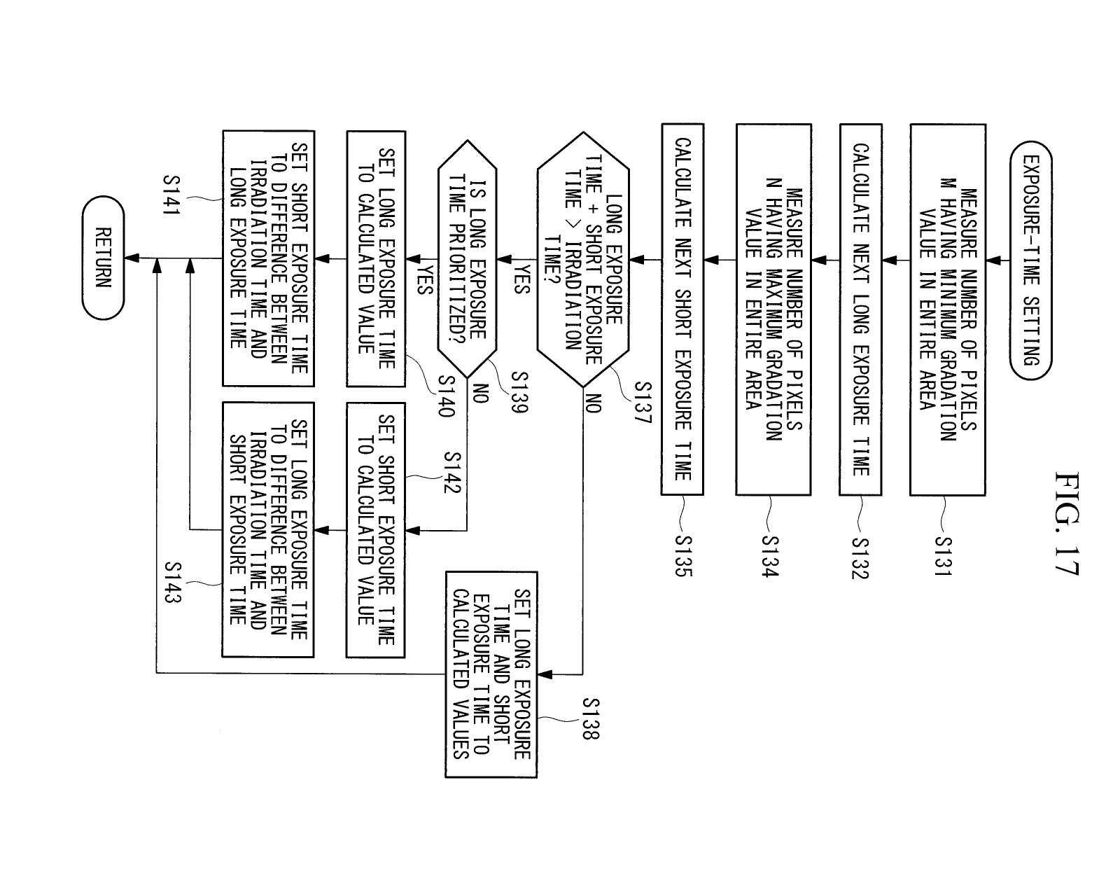

Note that, in this embodiment, as shown in FIG. 16, it is also possible to further provide an input unit 21 with which an observer selects which to prioritize: the resolution of overexposed highlights or the resolution of underexposed shadows.

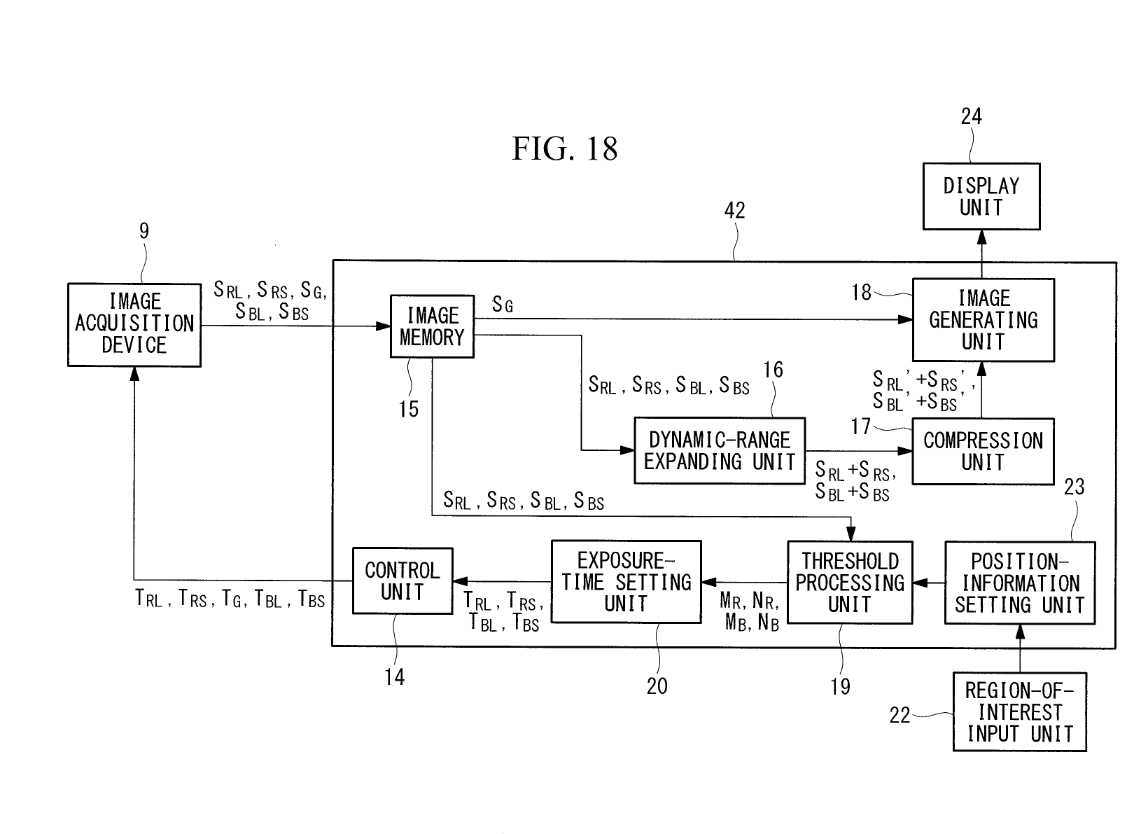

As a result of the above-described calculation of the next long exposure times T_{RL} and T_{BL} and the next short exposure times T_{RS} and T_{BS}, the sum T_{RL}+T_{RS} or T_{BL}+T_{BS} of the long exposure time and the short exposure time can exceed the irradiation time for the illumination light L_{R} or L_{B} per single irradiation. In such a case (YES in Step S137), as shown in FIG. 17, the exposure-time setting unit 20 sets the long exposure times T_{RL} and T_{BL} and the short exposure times T_{RS} and T_{BS} (Steps S138 to S143) according to one of the resolution of overexposed highlights and the resolution of underexposed shadows selected by using the input unit 21 (Step S139).

If resolution of underexposed shadows is prioritized (YES in Step S139), the exposure-time setting unit 20 preferentially sets the long exposure time T_{RL} (Step S140) and sets the short exposure time T_{RS} to the time obtained by subtracting the long short exposure time T_{RL} from the irradiation time (Step S141). If resolution of overexposed highlights is prioritized (NO in Step S139), the exposure-time setting unit 20 preferentially sets the short exposure time T_{RS} (Step S142) and sets the long exposure time T_{RL} to a time obtained by subtracting the short exposure time T_{RS} from the irradiation time (Step S143).

If the sum T_{RL}+T_{RS} of the long exposure time and the short exposure time is equal to or less than the irradiation time for the illumination light L_{R} (NO in Step S137), the next exposure times T_{RL} and T_{RS} are set as in the above-described Steps S133 and S136 (Step S138).

The same applies to the exposure times T_{BL} and T_{BS} for the B-image signals.

By doing so, in order to observe a dark area such as a concave portion in detail, the observer prioritizes the resolution of underexposed shadows, thereby making it possible to reliably observe an endoscope image 25 in which the dark area is clearly captured, and, in order to observe a bright area such as a convex portion in detail, the observer prioritizes the resolution of overexposed highlights, thereby making it possible to reliably observe an endoscope image 25 in which the bright area is clearly captured.

### Third Embodiment

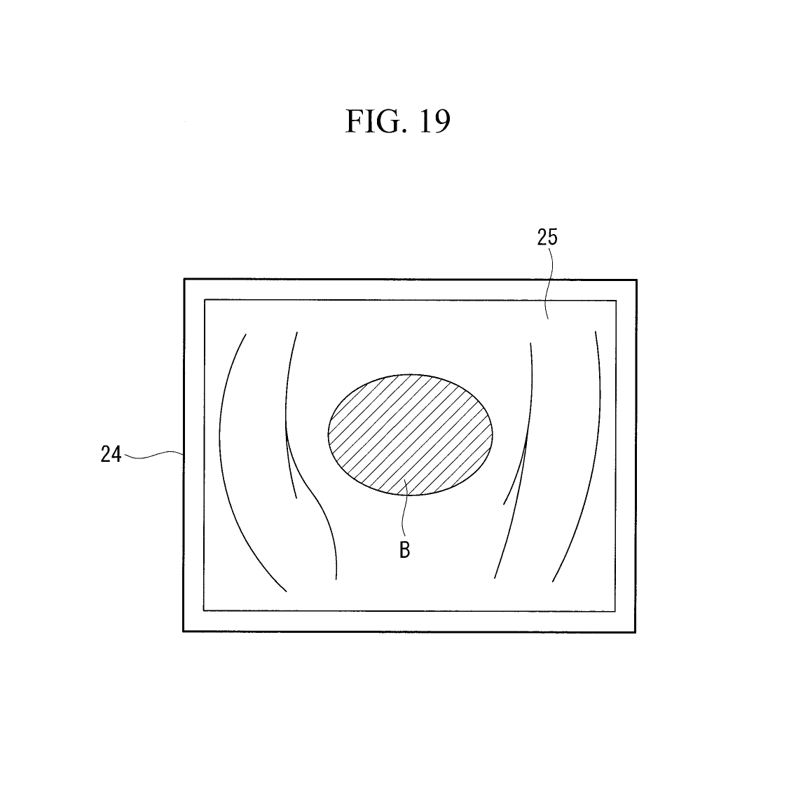

Next, an endoscope apparatus according to a third embodiment of the present invention will be described with reference to FIGS. 18 to 20.

The endoscope apparatus of this embodiment is obtained by modifying the endoscope apparatus of the second embodiment and differs from the endoscope apparatus of the second embodiment in that feedback control is performed on the exposure times T_{RL}, T_{RS}, T_{BL}, and T_{BS} for the next capturing of R-light L_{R} and B-light L_{B} on the basis of the distributions of the gradation values of pixels in a region of interest, instead of all pixels in the image signals S_{RL}, S_{RS}, S_{BL}, and S_{BS}.

Specifically, the endoscope apparatus of this embodiment is provided with an image processor 42 shown in FIG. 18, instead of the image processor 4. The configurations other than that of the image processor 42 are the same as those in the endoscope apparatus 1 of the first embodiment shown in FIG. 1.

As shown in FIG. 18, the image processor 42 is further provided with: a region-of-interest input unit (region-of-interest specifying unit) 22 and a position-information setting unit 23.

The region-of-interest input unit 22 is, for example, a pointing device, such as a stylus pen or a mouse, with which a position can be specified on an endoscope image displayed on the display unit 24. As shown in FIG. 19, the observer can specify, as a region of interest B, a desired region in the capture range of the endoscope image 25 displayed on the display unit 24, by using the region-of-interest input unit 22.

The position-information setting unit 23 obtains the position of the specified region of interest B from the region-of-interest input unit 22, converts the obtained position into the addresses of pixels in the endoscope image 25, and sends the addresses to the threshold processing unit 19.

The threshold processing unit 19 selects, among the pixels of the R-image signals S_{RL} and S_{RS} and the B-image signals S_{BL} and S_{BS} received from the image memory 15, the pixels in the region of interest B according to the addresses received from the position-information setting unit 23. Next, the threshold processing unit 19 compares the gradation values of the selected pixels with the thresholds α_{RL}, α_{RS}, α_{BL} and α_{BS}, thereby measuring the numbers of pixels M_{R}, N_{R}, M_{B} and N_{B}.

The exposure-time setting unit 20 determines the next long exposure times T_{RL} and T_{BL} on the basis of the numbers of pixels M_{R} and M_{B} and calculates the next short exposure times T_{RS} and T_{BS} on the basis of the numbers of pixels N_{R} and N_{B}. However, the maximum values of the numbers of pixels M_{R}, N_{R}, M_{B}, and N_{B} vary depending on the size of the region of interest B. Therefore, the exposure-time setting unit 20 multiplies the numbers of pixels M_{R}, N_{R}, M_{B}, and N_{B} by the ratio C_{B}/C of the total number of pixels C_{B} existing in the region of interest B with respect to the total number of pixels C in the entire endoscope image, thereby obtaining correction values M_{R}×C_{B}/C, N_{R}×C_{B}/C, M_{B}×C_{B}/C, and N_{B}×C_{B}/C for the numbers of pixels M_{R}, N_{R}, M_{B}, and N_{B}. Then, the exposure-time setting unit 20 calculates the next exposure times T_{RL}, T_{BL}, T_{RS}, and T_{BS} from the LUTs shown in FIGS. 11 and 12 by using the obtained correction values, instead of M or N.

Alternatively, the exposure-time setting unit 20 may hold a plurality of LUTs corresponding to the sizes of the region of interest B, i.e., the total numbers of pixels C_{B}. In the plurality of LUTs, the relationships between the numbers of pixels M_{R}, N_{R}, M_{B}, and N_{B} and the extension time or the reduction time have already been corrected according to the respective total numbers C_{B}. The exposure-time setting unit 20 can calculate the next exposure times T_{RL}, T_{BL}, T_{RS}, and T_{BS} by selecting an appropriate LUT according to the total number C_{B}.

Next, the operation of the thus-configured endoscope apparatus will be described.

The main routine in this embodiment is the same as the main routine in the second embodiment shown in FIG. 13, and the content of an exposure-time setting routine (Step S13) differs from that in the second embodiment.

According to the endoscope apparatus of this embodiment, as in the second embodiment, after two R-image signals S_{RL} and S_{RS} are obtained in Step S7, the exposure times T_{RL} and T_{RS} for obtaining next R-image signals S_{RL} and S_{RS} are set in the exposure-time setting routine S13.

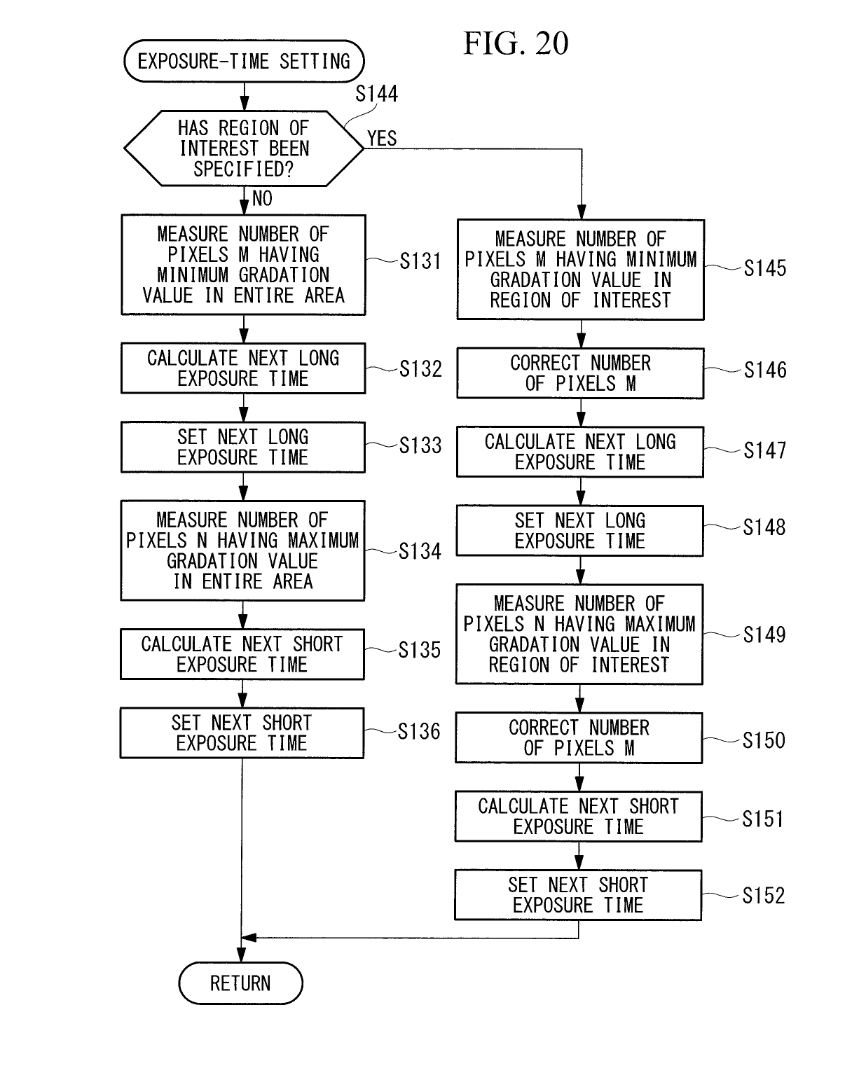

In the exposure-time setting routine S13, as shown in FIG. 20, first, it is determined whether the region of interest B has been specified (Step S144).

If the region of interest B has not been specified (NO in Step S144), the next exposure times T_{RL}, T_{RS}, T_{EL}, and T_{BS} are set according to the same procedure as that in the second embodiment (Steps S131 to S136).

If the region of interest B has been specified (YES in Step S144), the threshold processing unit 19 measures the number of pixels M_{R} that have the minimum gradation value 0 among the pixels that constitute the region of interest B (Step S145), corrects the measured number of pixels M_{R} according to the total number of pixels C_{B} in the region of interest R (Step S146), and calculates and sets the next long exposure time T_{RL} on the basis of the correction value M_{R}×C/C_{B} (Steps S147 and S148). Then, the threshold processing unit 19 measures the number of pixels N_{R} that have the maximum gradation value 255 among the pixels that constitute the region of interest B (Step S149), corrects the measured number of pixels N_{R} according to the total number of pixels C_{B} in the region of interest R (Step S150), and calculates and sets the next short exposure time T_{RS} on the basis of the correction value N_{R}×C/C_{B} (Steps S151 and S152). For B-image signals S_{BL} and S_{BS}, the next long exposure time T_{EL} and the next short exposure time T_{BS} are set in Steps S145 to S152, as in the R-image signals S_{RL} and S_{RS}.

In this way, according to this embodiment, the next exposure times T_{RL}, T_{RS}, T_{BL}, and T_{BS} are adjusted according to the presence or absence of overexposed highlights and underexposed shadows in the region of interest B, to which the observer particularly pays attention, in the endoscope image 25. Accordingly, there is an advantage that it is possible to acquire an endoscope image 25 that has high contrast in the region of interest B, thus making it possible to more accurately observe the region of interest B.

In the first to third embodiments, although capturing is performed two times for different exposure times during one irradiation period for each of R-light and B-light, instead of this, capturing may be performed three times or more. In this case, it is preferred that the exposure times for three captures be different from each other.

Furthermore, in the first to third embodiments, although the dynamic range is expanded in both of the R-image signal and the B-image signal, instead of this, the dynamic range may be expanded in only one of the R-image signal and the B-image signal. In this case, only the image signal of which the dynamic range is to be expanded needs to be acquired a plurality of times through a plurality of captures.

As a result, the following aspect is read by the above described embodiment of the present invention.

An aspect of the present invention provides an endoscope apparatus including: an illumination unit that sequentially radiates illumination light of three colors of red, green, and blue onto a subject; an image acquisition unit that acquires an image by capturing the illumination light reflected at the subject; a control unit that controls the image acquisition unit so as to perform capturing in synchronization with radiation of the illumination light of the three colors from the illumination unit, thereby causing the image acquisition unit to sequentially acquire component images of three colors of red, green, and blue; a dynamic-range expanding unit that generates an expanded component image in which the dynamic range is expanded, from the component image of at least one color other than green, among the component images of the three colors acquired by the image acquisition unit; and an image generating unit that generates a colored endoscope image by compositing the expanded component image of the at least one color, which is generated by the dynamic-range expanding unit, and the component images of the other colors, wherein the control unit controls the image acquisition unit so as to capture the illumination light of the at least one color a plurality of times for different exposure times, thereby causing the image acquisition unit to acquire a plurality of component images of the at least one color; and the dynamic-range expanding unit generates the expanded component image by compositing the plurality of component images of the at least one color.

According to the aspect, in synchronization with switching among red, green, and blue of illumination light radiated onto a subject from the illumination unit, the image acquisition unit performs capturing of the subject, thereby acquiring component images of three colors, and the image generating unit generates an RGB-format endoscope image from the acquired component images of the three colors.

In this case, in capturing of illumination light of red or/and blue, the control unit causes the image acquisition unit to perform capturing of illumination light of the same color a plurality of times for different exposure times, thereby acquiring a plurality of component images having different brightness.

The dynamic-range expanding unit composites the plurality of component images of the same color having different brightness, thereby generating a red expanded component image or/and a blue expanded component image having a wider dynamic range than the dynamic range of a green component image. In this way, by using the red component image or/and the blue component image having a wide dynamic range, it is possible to acquire an endoscope image in which the difference in color, in particular, red or/and blue, in living tissue is correctly reproduced.

In the above-described aspect, the control unit may control the image acquisition unit so as to capture each of the illumination light of red and the illumination light of blue a plurality of times for different exposure times and may control the exposure times for capturing the illumination light of red and the exposure times for capturing the illumination light of blue, independently from each other.

By doing so, the dynamic ranges of the red expanded component image and the blue expanded component image are controlled independently from each other, thus making it possible to acquire an endoscope image having higher color reproducibility in living tissue.

The above-described aspect may further include an exposure-time setting unit that sets exposure times for next capturing of the illumination light of the at least one color performed a plurality of times, on the basis of the distribution of gradation values of the plurality of component images of the at least one color.

The distribution of gradation values is biased toward a minimum gradation value side when the exposure time is insufficient, and the distribution of gradation values is biased toward a maximum gradation value side when the exposure time is excessive. The exposure-time setting unit determines excess or insufficiency of the exposure time on the basis of the distribution of gradation values, sets a longer exposure time for next capturing when the exposure time is insufficient, and sets a shorter exposure time for next capturing when the exposure time is excessive. Accordingly, it is possible to acquire a component image having appropriate contrast, in the next capturing.

The above-described aspect may further include a region-of-interest specifying unit that specifies a region of interest in a capture range of the component image captured by the image acquisition unit, wherein the exposure-time setting unit may set exposure times for next capturing of the illumination light of the at least one color performed a plurality of times, on the basis of the distribution of gradation values in the region of interest specified by the region-of-interest specifying unit, among the plurality of component images of the at least one color.

By doing so, the dynamic range of a red expanded component image or/and a blue expanded component image is optimized on the basis of the color and the brightness of the region of interest. Therefore, it is possible to ensure high color reproducibility in the region of interest in the endoscope image.

### REFERENCE SIGNS LIST

1 endoscope apparatus
2 insertion portion
3 illumination unit (illumination unit)
4, 41, 42 image processor
5 illumination lens
6 objective lens
7, 11, 12 condensing lens
8 light guide
9 image acquisition device (image acquisition unit)
10 light source
13 color filter set
14 control unit
15 image memory
16 dynamic-range expanding unit
17 compression unit
18 image generating unit
19 threshold processing unit
20 exposure-time setting unit
21 input unit
22 region-of-interest input unit (region-of-interest specifying unit)
23 position-information setting unit
24 display unit
25 endoscope image

1. An endoscope apparatus comprising:
an illumination unit that sequentially radiates illumination light of three colors of red, green, and blue onto a subject;
an image acquisition unit that acquires an image by capturing the illumination light reflected at the subject;
a control unit that controls the image acquisition unit so as to perform capturing in synchronization with radiation of the illumination light of the three colors from the illumination unit, thereby causing the image acquisition unit to sequentially acquire component images of three colors of red, green, and blue;
a dynamic-range expanding unit that generates an expanded component image in which the dynamic range is expanded, from the component image of at least one color other than green, among the component images of the three colors acquired by the image acquisition unit; and
an image generating unit that generates a colored endoscope image by compositing the expanded component image of the at least one color, which is generated by the dynamic-range expanding unit, and the component images of the other colors,
wherein the control unit controls the image acquisition unit so as to capture the illumination light of the at least one color a plurality of times for different exposure times, thereby causing the image acquisition unit to acquire a plurality of component images of the at least one color; and
the dynamic-range expanding unit generates the expanded component image by compositing the plurality of component images of the at least one color.

2. An endoscope apparatus according to claim 1, wherein the control unit controls the image acquisition unit so as to capture each of the illumination light of red and the illumination light of blue a plurality of times for different exposure times and controls the exposure times for capturing the illumination light of red and the exposure times for capturing the illumination light of blue, independently from each other.

3. An endoscope apparatus according to claim 1, further comprising an exposure-time setting unit that sets exposure times for next capturing of the illumination light of the at least one color performed a plurality of times, on the basis of the distribution of gradation values of the plurality of component images of the at least one color.

4. An endoscope apparatus according to claim 3, further comprising a region-of-interest specifying unit that specifies a region of interest in a capture range of the component image captured by the image acquisition unit,
wherein the exposure-time setting unit sets exposure times for next capturing of the illumination light of the at least one color performed a plurality of times, on the basis of the distribution of gradation values in the region of interest specified by the region-of-interest specifying unit, among the plurality of component images of the at least one color.
